(19) United States
(12) Patent Application Publication
(10) Pub. No.: US 2004/0137522 A1
(43) Pub. Date: Jul. 15, 2004

(54) Genes and proteins altering Tau-related neurodegeneration

(75) Inventors: Mel B. Feany, Cambridge, MA (US); Joshua M. Shulman, Boston, MA (US)

Correspondence Address: Fitch, Even, Tabin & Flannery, Suite 401L, 1801 K Street, N.W., Washington, DC, 20006-1201, US

(73) Assignee: The Brigham and Women's Hospital, Inc.

(21) Appl. No.: 10/655,601

(22) Filed: Sep. 5, 2003

Related U.S. Application Data
(60) Provisional application No. 60/408,877, filed on Sep. 9, 2002.

(51) Int. Cl. C12Q 1/68; G01N 33/53; G01N 33/567; A61K 38/17; C07K 14/705; C07H 21/04

(52) U.S. Cl. 435/7.1; 514/12; 530/350; 536/23.5; 435/69.1; 435/320.1; 435/325; 435/6

(57) ABSTRACT

The present invention is based upon the identification of several genes that either enhance or suppress Tau-related neurodegeneration when expressed. These may be used in diagnostic assays and in assays designed to find factors that may be used to treat neurological diseases, such as Alzheimer's disease.

### CROSS REFERENCE TO RELATED APPLICATIONS

This application claims priority to U.S. provisional application No. 60/408,877, filed on Sep. 9, 2002, which is incorporated in its entirety herein by reference.

### STATEMENT OF GOVERNMENT SUPPORT

The work leading to this invention was supported by one or more grants from the U.S. Government. The U.S. Government therefore has certain rights in the invention.

### FIELD OF THE INVENTION

The present invention is directed to a variety of genes and proteins that have been identified as enhancing or suppressing Tau-related neurodegeneration. These may be used in assays designed to identify agents useful in the treatment of, inter alia, Alzheimer's disease. In addition, vectors encoding factors suppressing Tau-related neurodegeneration may be administered to patients to treat or prevent disease.

### BACKGROUND OF THE INVENTION

Alzheimer's disease is the most common neurodegenerative disorder and causes progressive memory loss and, ultimately, severe cognitive dysfunction and death. Dementia is accompanied pathologically by neuronal loss, and the diagnostic hallmarks of Alzheimer's disease: amyloid plaques and neurofibrillary tangles. Plaques are extracellular accumulations of amyloid-β (Aβ), a proteolytic fragment of the amyloid precursor protein, whereas intracellular neurofibrillary tangles consist of abnormally phosphorylated, aggregated Tau. Hyperphosphorylated and aggregated Tau is also the primary neuropathologic manifestation of a less common group of neurodegenerative diseases that includes frontotemporal dementia and related disorders, known as “tauopathies.” Genetic evidence for a causative role of Tau in neurodegeneration has been provided by the recent demonstration that dominant mutations in the tau gene cause frontotemporal dementia and Parkinsonism linked to chromosome 17 (FTDP-17)¹⁻³. Although similar mutations have not been found in Alzheimer's disease, the appearance and anatomic distribution of neurofibrillary pathology correlates well with neuronal loss and cognitive dysfunction, suggesting that wild-type Tau may directly contribute to neuronal degeneration.^{4,5}

The mechanism of Tau neurotoxicity in Alzheimer's disease and related disorders has been the subject of extensive investigation, and altered protein phosphorylation has been implicated as a major determinant of Tau toxicity.⁶ Tau protein purified from the brains of patients with Alzheimer's disease is hyperphosphorylated.⁷⁻⁹ In addition, antibodies recognizing selected Tau phosphoepitopes show specific staining of Tau from Alzheimer's disease brain tissue.¹⁰⁻¹² In general, hyperphosphorylation decreases the affinity of Tau for microtubules and increases homotypic interactions, thus promoting aggregation.^{13,14} Several tau missense mutations associated with FTDP-17 have similar effects on microtubule binding and aggregation, suggesting that these changes might form the basis of Tau neurotoxicity. ^{1,15} Such observations have motivated extensive efforts to identify the kinases responsible for phosphorylation of Tau in Alzheimer's disease, and a number of candidates have been identified. In some cases, alterations in the expression, localization, or activity of candidate kinases has been observed in the brains of patients with Alzheimer's disease.¹⁶ However, experimental proof linking Tau hyperphosphorylation, or increased activity of particular kinases, to neurodegeneration in vivo has not been demonstrated.

Drosophila models have been successfully developed for anumber of neurodegenerative diseases, and these systems are now being exploited to dissect the genetic pathways underlying disease pathogenesis.¹⁷ A major advantage of Drosophila as a model system is the ability to conduct unbiased genetic screens for enhancers and suppressors of neurodegeneration in vivo. This approach has been successfully applied to Drosophila models of the polyglutamine repeat disorders, which include Huntington's disease and spinocerebellar ataxia.^{18,19} Such genetic screens, as well as candidate-based approaches, have revealed that mutations in heat shock proteins and components of the ubiquitin proteasome degradation pathway can modulate polyglutamine toxicity in vivo.²⁰ Molecular chaperones have been similarly implicated as modulators of neurodegeneration in a Drosophila model of Parkinson's disease.²¹ These results suggest that the misfolding, impaired degradation, and abnormal aggregation of proteins are key determinants in the pathogenesis of neurodegenerative disease.²²

A Drosophila model of tauopathy has recently been developed that allows the determinants of Tau toxicity to be studied in vivo. ²³ Expression of human tau in the Drosophila brain recapitulates several features of human tauopathies, including age-dependent neurodegeneration, early death, abnormally phosphorylated and folded Tau, and increased toxicity of mutant versus wild-type Tau. This model should allow for the identification of the genes that contribute to Tau-related neurodegenerative diseases and which may be used in the development of new therapies and diagnostic assays.

### SUMMARY OF THE INVENTION

The present invention is based upon experiments in which gene mutations were screened to identify those that enhance or suppress toxicity in a Drosophila model of tauopathy. This procedure revealed a cohort of genes that appear to be either suppressors or enhancers of toxicity and which may be used in diagnostic assays and in assays designed to identify potential therapeutic agents. Vectors which code for suppressors may also be used in gene therapy procedures for the treatment of patients.

The genes (and proteins) identified fall into two categories: a) modulators of Tau-related neurotoxicity that have not been previously described; and b) modulators that were known structurally but whose effects in relation Tau-related toxicity were not.

A) Previously Undisclosed Modulators of Tau-Related Neurotoxicity

The invention is concerned with several genes and proteins that have not been previously described and which act as suppressors of Tau-related neurotoxicity. These include all those shown in Table 1.

TABLE 1
New Modulators of Tau-Related Neurotoxicity

| Drosophila | Apparent effect on | | Human | Sequence ID |
|---|---|---|---|---|
| Gene | Toxicity | Apparent Function | Homolog | Number* |
| wunen | suppressor | phosphatidic acid | PPAP2A | SEQ ID NO: 1 |
| | | phosphatase | | |
| | suppressor | | PPA2B | SEQ ID NO: 2 |
| CG10082 | enhancer | inositol hexokisphos- | IHPK1 | SEQ ID NO: 3 |
| | | phate kinase | | |
| | enhancer | | IHPK3 | SEQ ID NO: 4 |
| | enhancer | | IHPK2 | SEQ ID NO: 5 |
| CG5859 | suppressor | unknown | FLJ20530 | SEQ ID NO: 6 |
| CG3735 | suppressor | unknown | DJ434014.5 | SEQ ID NO: 7 |
| CG11166 | suppressor | unknown | EAF1 | SEQ ID NO: 8 |
| CG7231 | suppressor | unknown | Loc127424 | SEQ ID NO: 9 |
| CG10927 | suppressor | unknown | Loc113179 | SEQ ID NO: 10 |
| furry | enhancer | unknown | KIAA0826 | SEQ ID NO: 11 |

*refers to the human sequence

In its first aspect, the invention is directed to a substantially pure protein consisting essentially of an amino acid sequence corresponding to any of SEQ ID NOs:1-11. As used herein, the term “substantially pure” refers to a protein or polynucleotide that has been separated from other accompanying biological components and which typically comprises at least 85% of a sample, with greater percentages being preferred. Many means are available for assessing the purity of a protein or nucleic acid within a sample, including analysis by polyacrylamide gel electrophoresis, chromatography and analytical centrifugation.

The phrase “consisting essentially of” as used herein has its ordinary accepted meaning in patent law. Specifically, the phrase limits the scope of an invention to the specific materials or steps recited and other, additional, materials or steps provided that the latter do not materially affect the basic and novel characteristics of the invention. As used in the present instance, the phrase allows alterations either within or outside of a DNA sequence to the extent that the changes do not result in a DNA that is not structurally novel or that no longer encodes a protein which modulates Tau neurotoxicity as measured using the Drosophila model described herein. A similar meaning applies with respect to the other polynucleotides and proteins below, i.e., structural variations are allowed to the extent that they do not result in a polynucleotide or polypeptide that is no longer structurally novel or that no longer maintains its ability to suppress or enhance Tau neurotoxicity.

The invention is also directed to a process for producing an antibody that binds preferentially to one of the modulator proteins described above. “Preferential binding” refers to antibodies that have at least a 100-fold greater affinity for the suppressor than for any other protein normally found in the human or Drosophila. Preferably, such antibodies also have a 100-fold greater affinity for the human modulator than for similar proteins derived from other species. The invention encompasses a process for producing these antibodies by administering purified protein as defined by SEQ ID NOs:1-11 to an animal capable of antibody production. The protein should be administered at a dosage sufficient to induce antibody formation in the animal. The antibodies obtained in this manner may be used in radioligand binding assays designed to determine the amount of protein present in a biological sample. These assays may be used to determine whether an individual has abnormally elevated or lowered amounts of protein present in serum or other biological material relative to that seen in the population as a whole. Abnormal levels are an indication that the person has or is at an increased risk of developing a Tau-related neurodegenerative condition such as Alzheimer's disease. PCR analysis can also be performed to determine whether mutations have occurred that alter the structure of an enhancer or suppressor protein. Such mutations would suggest that an individual is at increased risk of having or developing a Tau-related neurodegenerative condition such as Alzheimer's disease.

In another aspect, the invention is directed to a substantially pure polynucleotide encoding one of the Tau-related proteins described above. It also encompasses vectors having a distinct coding element (i.e., a region with a coding sequence) which is identical to the sequence of any one of these polynucleotides. Preferably the vectors are “expression vectors” in which the coding element is operably linked to a promoter. The term “operably linked” refers to genetic elements that are joined in a manner that enables them to carry out their normal function. For example, a gene is operably linked to a promoter when its transcription is under the control of the promoter and the transcript produced is correctly translated into the protein normally encoded by the gene.

The invention also includes host cells transformed with one or more of the expression vectors described above. The term “host cell” encompasses essentially any type of cell capable of expressing the gene. However, it does not include human cells that have been transfected in vivo and which have not been removed from the person in which they were originally present.

In another aspect, the invention is directed to an assay for screening a test compound for its ability to increase the expression of a DNA that suppresses Tau-related neurotoxicity. The assay is performed by incubating cells that express one of the polynucleotides described above in the presence of the test compound and measuring the expression of the gene using standard techniques. The expression measured is compared with the expression of the same gene in control cells that are treated in a similar manner but which are not exposed to the test compound. An increase in expression in the presence of the test compound is an indication that it should be useful in suppressing tau-related neurotoxicity.

The invention also encompasses methods of treating a patient for a Tau-related neurodegenerative condition, such as Alzheimer's disease, by administering an effective amount of a nucleic acid sequence encoding one or more of the suppressor proteins described. The term “effective amount” means that sufficient nucleic acid is delivered for it to be taken up by cells, and for there to then be sufficient expression of the suppressor to reduce toxicity as evidenced by an improvement in one or more symptoms characteristic of the disease. Methods for the in vivo delivery of nucleic acids are well known in the art and may involve the use of viral vectors (adenoviral vectors), chemical agents promoting in vivo cell transformation (e.g, liposomes), or the direct administration of naked DNA. Alternatively, cells may be removed from a patient, transformed and then re-implanted. In addition to being useful for the treatment of Alzheimer's disease, it is believed that these procedures will also be of value in treating other Tau-related neuopathies such as PSP; CBD; Pick's disease and FTDP-17. Also, by determining whether a patient carries a mutated form of one of the proteins discussed above.

B) Additional Modulators of Tau-Related Neurotoxicity

In addition to the modulators of Tau-related neurotoxicity described above, several additional factors, already known in the art but apparently not previously associated with effects on Tau-related neurotoxicity, were found to act as modulators. These are shown in Table 2.

TABLE 2
Additional Modulators of Tau-Related Neurotoxicity

| | Apparent | | | |
|---|---|---|---|---|
| Drosophila | effect on | Mammalian | Human | |
| Gene | Toxicity | Homolog/Function | Homolog | Reference* |
| CG10967 | suppressor | Unc-51 | ULK1 | Kuroyanagi, et al., |
| | | serine/threonine | (12q24.3) | Genomics. 1998 Jul |
| | | kinase | | 1; 51(1): 76-85. |
| | | | ULK2 | Yan, et al., Oncogene. 1999 |
| | | | (17p11.2) | Oct 21; 18(43): 5850-9. |
| CG14217 | enhancer | Tao1 serine/threonine | TAO1 (16) | Hutchison, et a1., J Biol |
| | | kinase | | Chem. 1998 Oct |
| | | | | 30; 273(44): 28625-32. |
| center divider | enhancer | TESK1 | TESK1 (9p13) | Toshima, et al., J. Biol. |
| | | serine/threonine | | Chem. 1995 Dec |
| | | kinase | | 29; 270(52): 31331-7. |
| | | | TESK2 (1p32) | Rosok, et al., Genomics. |
| | | | | 1999 Oct 1; 61(1): 44-54. |
| CG5166 | enhancer | Ataxin-2 | SCA2 (12q24) | Imbert, et al., Nat Genet. |
| | | | | 1996 Nov; 14(3): 285-91. |
| dfxr | enhancer | Fragile-X | FXR1 (3q28) | Tamanini, et al., Hum Mol |
| | | | FXR2 | Genet. 1997 Aug; 6(8): 1315-22. |
| | | | (17p13.1) | |
| | | | FMR1 (Xq27.3) | Verkerk, Cell. 1991 May |
| | | | | 31; 65(5): 905-14. |
| cheerio | enhancer | filamin | FLNA (Xq28) | Chakarova, et al. Hum |
| | | | FLNB (3p14.3) | Genet. 2000 |
| | | | FLNC (7q32-q35) | Dec; 107(6): 597-611 |
| orbit/mast | enhancer | microtubule | CLASP1 | Akhmanova, et al., Cell. |
| | | associated protein | (2q14.2) | 2001 Mar 23; 104(6): 923-35. |
| | | | CLASP2 | |
| | | | (3p22.2) | |
| CG8487 | enhancer | sec7 GEF | GBF1 (10q24) | Achstetter, et al., J Biol |
| | | | | Chem. 1988 Aug |
| | | | | 25; 263(24): 11711-7. |
| dally | enhancer | glypican | GPC5 (13q32) | Veugelers, et al., Genomics. |
| | | | | 1997 Feb 15; 40(1): 24-30. |

The same assays described in section A above may be used in connection with any of the modulators in Table 2. Specifically, a test compound may be screened for its ability to increase or decrease the expression of a gene that modulates Tau-related neurotoxicity by incubating cells that express the gene in the presence of the test compound and measuring expression using standard techniques. The expression measured is compared with the expression of the same gene in control cells that are treated in a similar manner but which are not exposed to the test compound.

Diagnostic assays may be performed in which the amount of one or more of the proteins listed in Table 2 is measured in a patient's serum or other biological fluid (e.g., cerebrospinal fluid). Abnormally elevated or reduced levels relative to that seen in a control group consisting, for example, of the general population or a group known to be disease free would be an indication of the presence of a Tau-related neurodegenerative condition. Protein levels may be determined using standard immunological assays.

Tau-related neuropathies may be treated by administering an effective amount of a nucleic acid sequence encoding the CG10967 suppressor protein described above. Thus, the same methods described above in connection with previously undisclosed suppressors may be used for the suppressor set forth in Table 2. Any of the methods know for the in vivo delivery of nucleic acids may be employed for this purpose.

### DETAILED DESCRIPTION OF THE INVENTION

The present invention is directed to a variety of genes that encode proteins capable of contributing to Tau-related neurotoxicity. Some of the genes, when expressed, suppress toxicity, whereas others enhance toxicity. Suppressors are of particular interest because methods which increase their cellular activity should help to alleviate symptoms associated with several neurological diseases, including Alzheimer's disease. There are essentially two ways in which activity can be increased. One way is to contact cells with an agent that promotes their expression. Thus, one use of the genes is to identify these agents. The assays may measure changes in mRNA levels (e.g., using PCR or blotting techniques) or the assays may measure levels of gene products using immunoassays. Antibodies to the encoded proteins and the proteins themselves are of value in the latter type of assay.

The second way in which the cellular activity of suppressor genes may be increased is by introducing nucleic acids that increase suppressor gene expression. For example, cells may be transfected or infected with an expression vector containing a region coding for the suppressor gene product operably linked to a promoter active in the cells. Alternatively, DNA may be introduced that is designed to incorporate a regulatory element increasing expression close enough to the endogenous gene to increase its expression. This may be accomplished using techniques of homologous recombinaztion that have been described in the art.

In addition to genes that suppress Tau-related neurotoxicity, several genes have been identified that enhance toxicity when they are expressed. These cannot be used directly as therapeutic agents but agents that inhibit their expression or activity may be of therapeutic value.

Assays in which a sample of biological fluid is removed from a subject and tested to determine the level of a Tau-related protein may be used diagnostically to help determine whether a person has a Tau-related neurodegenerative condition such as Alzheimer's disease. Preferably this is accomplished using an immunoassay and comparing the results for a test subject with those from a control group. In general abnormally low levels of a suppressor protein or abnormally elevated levels of an enhancer protein would be indicative of the presence of disease. Mutational analyses to may also be helpful in this regard. For example, individuals carrying mutations in a suppressor protein would be at an increased risk of developing a Tau-related neurodegenerative condition. Diagnostic assays designed to determine the activity of the genes, or their products, should also be useful in helping to diagnose a Tau-related neurodegenerative condition. The assays could be performed on cells derived from a patient at biopsy and would involve either measuring mRNA levels, protein levels or protein activity.

Factors that inhibit the expression of enhancer genes or that reduce the activity of the proteins that they encode should be useful therapeutic agents. Similarly, factors that increase the expression or activity of one or more suppressors should also be of value. In order to identify these agents, the Tau-related genes or proteins may be used in the screening assays described herein.

I. Protein and Nucleic Acid Sequences

The structure of the suppressor and enhancer proteins of the present invention are shown in the enclosed sequence listing or described in the publications set forth in Table 2 (such publications being hereby incorporated by reference). It will be understood that the invention encompasses not only sequences identical to those shown but also sequences that are essentially the same as evidenced by their retaining the same basic structural and functional characteristics. For example, techniques such site directed mutagenesis may be used to introduce variations into a protein structure. Variations introduced by this or other similar methods are encompassed by the invention provided that the resulting polypeptide retains its biological characteristics with respect to Tau-related neurotoxicity and remains structurally distinct from any other protein known in the art.

Many methods are available for producing and isolating DNA or proteins, any of which may be used for obtaining the suppressors and enhancers described herein (see e.g., Sambrook, et al., Molecular Cloning: A Laboratory Manual, 2^{nd} ed., Cold Spring Harbor Press (1989)). For example, one method is to screen a cDNA library that has been prepared by reverse transcribing mRNA isolated from tissues or cells that express the gene. The library may be screened using probes synthesized based upon the sequences shown in the Sequence Listing. Alternatively, amplification of the desired sequences may be achieved using the polymerase chain reaction (“PCR”) of reverse transcribed RNA. Primers for PCR may be constructed using the sequences shown herein. Confirmation that the correct sequence has been amplified may be obtained by sequencing amplification products. Finally, both nucleic acids and proteins may be obtained using standard chemical synthetic methods.

II. Production of Suppressor or Enhancer Protein

Apart from the chemical synthesis of protein, production may, if desired, be carried out by recombinant means. Expression may be induced in a host cell by transforming it with an appropriate expression vector. The vector should contain transcriptional and translational signals recognizable by the host, together with the desired structural sequence in an operable linkage, i.e., nucleotides encoding a suppressor protein should be positioned such that regulatory sequences present in the vector control the synthesis of mRNA and a protein having the correct sequence is ultimately produced.

Preferably, nucleic acid encoding suppressor or enhancer protein is expressed in eukaryotic cells, especially mammalian cells. Such cells are capable of promoting post-translational modifications necessary to ensure that the recombinant protein is structurally and functionally the same as that found in nature. Examples of mammalian cells known to provide post-translational modification of cloned proteins include, inter alia, NIH-3T3 cells, CHO cells, HeLA cells, LM (tk−) cells, and the like. Eukaryotic promoters known to control recombinant gene expression are preferably utilized and may include that of the mouse metallothionein I gene, the TK promoter of Herpes virus, the CMV early promoter and the SV40 early promoter.

Expression vectors may be introduced into host cells by any method known in the art (e.g., calcium phosphate precipitation, micro-injection, electroporation, or viral transfer) and cells expressing recombinant protein can be selected by established techniques. Confirmation of expression may be obtained by PCR amplification using primers selected from the sequences shown.

Recombinant protein may be purified using standard techniques well known in the art.

Such techniques may include filtration, precipitation, chromatography and electrophoretic methods. Purity can be assessed by performing electrophoresis on a polyacrylamide gel and visualizing proteins using standard staining methodology. Protein obtained in this way may be used in the generation of antibodies or in assays as described below.

III. Antibodies to Enhancer or Suppressor Polypeptides

The present invention includes antibodies raised against the suppressor and enhancer proteins described herein. The process for producing such antibodies may involve injecting protein into an appropriate animal or injecting short antigenic peptides made to correspond to different regions of the protein. These peptides should be at least 5 amino acids in length and should, preferably, be selected from regions believed to be unique to the tumor suppressor protein. Methods for generating and detecting antibodies are well know in the art and are taught by such references as: Harlow, et al., Antibodies, A Laboratory Manual, Cold Spring Harbor Laboratory, N.Y. (1988); Klein, Immunology: The Science of Self-Nonself Discrimination, (1982); and Campbell, “Monoclonal Antibody Technology”, in Laboratory Techniques in Biochemistry and Molecular Biology, (1984).

The term “antibody”, as used herein, is meant to include intact molecules as well as fragments that retain their ability to bind antigen, such as Fab and F(ab′)₂ fragments. The term “antibody” is also defined as referring to both monoclonal antibodies and polyclonal antibodies. Polyclonal antibodies are derived from the sera of animals immunized with a tumor suppressor antigen. Monoclonal antibodies to a suppressor can be prepared using hybridoma technology, as taught by such references as: Hammerling, et al., in: Monoclonal Antibodies and T-Cell Hybridomas, Elsevier, N.Y., pp. 563-681 (1981). In general, this technology involves immunizing an immunocompetent animal, typically a mouse, with either intact protein, or a fragment derived therefrom. Splenocytes are then extracted from the immunized animal and are fused with suitable myeloma cells, such as SP₂O cells. Thereafter, the resulting hybridoma cells are selectively maintained in HAT medium and then cloned by limited dilution (Wands, et al., Gastroenterology 80:225-232 (1981)). Cells obtained through such selection are then assayed to identify clones which secrete antibodies capable of binding suppressor protein.

Antibodies or fragments of antibodies of the invention may be used to detect the presence of protein in any of a variety of immunoassays. These assays may be useful in determining whether a particular individual shows abnormally low levels of suppressor protein (or high levels of enhancer) and may therefore be susceptible to the development of Tau-related neurodeneration. For example, antibodies may be used in radioimmunoassays or in immunometric assays, also known as “two-site” or “sandwich” assays. In a typical immunometric assay, a quantity of unlabeled antibody is bound to a solid support that is insoluble in the fluid being tested. Following the initial binding of antigen to immobilized antibody, a quantity of detectably labeled second antibody (which may or may not be the same as the first) is added to permit detection and/or quantitation of bound antigen (see, e.g. Radioimmune Assay Method, Kirkham, et al., ed. pp. 199-206, E&S Livingstone, Edinburgh (1970)). Many variations of these types of assays are known in the art and may be employed for the detection of suppressors or enhancers.

Antibodies to protein may also be used in purification procedures (see generally, Dean et al., Affinity Chromatography A Practical Approach, IRL Press (1986)). Typically, these procedures involve immobilizing antibody on a chromatographic matrix such as Sepharose, 4B. The matrix is then packed into a column and the preparation containing suppressor protein is passed through under conditions that promote binding, e.g, under low salt conditions. The column is then washed and protein is eluted using a buffer that promotes dissociation from antibody, e.g., in a buffer having an altered pH or salt concentration. The eluted protein may be transferred into a buffer, for example via dialysis, and thereafter either stored or used directly.

Antibodies may also be used in Western blotting for the detection of suppressor protein in a sample. Shifts in the position of bands obtained in Western blots may be indicative of mutations in a protein.

IV. Therapeutic Methods

Therapeutic methods may involve either the administration of polypeptide or the administration of nucleic acids that encode the polypeptide. In the latter case, oligonucleotides designed for the expression of suppressor may be administered directly to patients or, alternatively, cells from patients may be removed, transfected and then re-implanted. The in vivo transfection of cells has been known for many years and may be accomplished using viral vectors (see e.g. U.S. Pat. No. 6,020,191); liposomes (see e.g., Nicolau, Meth. Enzymol 149:157-176 (1987)); DNA complexed to agents that facilitate cellular uptake (see e.g., U.S. Pat. No. 5,264,618; WO 98/14431); or even by simply injecting naked DNA (see e.g., U.S. Pat. No. 5,693,622). Administration may be repeated as is necessary until a positive therapeutic effect is observed. Administration may be continued thereafter based upon clinical considerations.

As an alternative to gene therapy, suppressor polypeptide may be directly administered to a patient. In order to preserve activity, the polypeptide should be administered parentally, with administration by injection being preferred. The dosage administered to a patient will be determined by the attending physician based upon clinical considerations and using methods well known in the art.

Suppressor polypeptides may be administered in either a single or multiple dosage regimen and may be given either alone or in conjunction with other therapeutic agents. Parenteral compositions may be used for intravenous, intraarterial, intramuscular, intraperitoneal, intracutaneous, or subcutaneous delivery. These preparations may be made using conventional techniques and may include isotonic saline, water, polyglycols, Ringer's solution, etc. All dosage forms may be prepared using methods that are standard in the art (see e.g., Remington's Pharmaceutical Sciences, 16 edition, A. Oslo editor, Easton, Pa. (1980)).

### EXAMPLES

I. Summary

In Alzheimer's disease and related disorders, the microtubule-associated protein Tau is abnormally hyperphosphorylated and aggregated into neurofibrillary tangles, and mutations in the tau gene cause familial frontotemporal dementia. In order to dissect the molecular mechanisms responsible for Tau-induced neurodegeneration, we have conducted an unbiased, forward genetic modifier screen in a Drosophila model of tauopathy. Kinases and phosphatases comprised the major class of modifiers recovered, and several candidate Tau kinases are also shown to enhance Tau toxicity. These results demonstrate that kinases and phosphatases control Tau-induced neurodegeneration in vivo. Additional genetic modifiers implicate the neuronal cytoskeleton and apoptosis as important determinants of Tau neurotoxicity. A direct comparison of modifiers among Drosophila models of neurodegenerative diseases reveals that the genetic pathways controlling Tau and polyglutamine toxicity are largely distinct, despite some clinical and pathologic similarities among neurodegenerative disorders.

II. Methods

Genetics

UAS-Tau^{WT}, UAS-Tau^{R406W}, and UAS-Tau^{V337M} transgenic Drosophila lines have been described previously.²³ EP strains and some mutant stocks were obtained from the Bloomington Drosophila Stock center and from Exelixis. The following mutations and transgenic strains were used: par-l^{W3} and UAS-parl⁷⁸; UAS-stg⁷⁹; th^{SL} and UAS-th⁴⁴; GMR-diapl and th^{i5C8 43}; Stg⁰¹²³⁵ and twe^{k08310 80}; UAS-wun2-myc and Df(2R)w73-l⁸¹; aop¹⁸²; UAS-aop^{WT}, GMR-yan^{WT}, and SEV-yan^{4CT 83}, UAS-dally⁸⁴; UAS-PKAmC⁸⁵; UAS-PKAcF⁸⁶; UAS-hep⁶³; UAS-sgg⁸⁷; UAS-cdk5-FLAG⁸⁸; UAS-p35⁸⁹; Pros^{1 90}.

EP modifiers of the Tau-induced rough eye phenotype were selected on the basis of their ability to modify the phenotype of UAS-TauV^{337M}/+; GMR-GAL4/+animals. Candidate modifiers were also tested for their ability to modify the same genotype. Fly cultures and crosses were routinely carried out at 25° C. The UAS/GAL4 expression system is temperature dependent, with increased expression at higher temperatures. In the case of candidate kinases that produced a rough eye when expressed with GMR-GAL4 alone at 25° C., additional crosses were performed at 17° C. All EP modifiers were also tested for their ability to modify the rough eye phenotype of UAS-Tau^{WT}, GMR-GAL4/+, and UAS-Tau^{R406W}, GMR-GAL4/+. The ability of modifiers to alter neurodegeneration in the brain was determined in the genotype elav-GAL4/+; UAS-Tau^{R460W}/+. Effects of modifiers in a polyglutamine model were tested in the UAS-SCA1-82/+; GMR-GAL4/+genotype.

Anatomic Analysis

Adult flies were fixed in formalin, and embedded in paraffin. Histologic analysis was performed on 4 μm sections stained with hematoxylin and eosin. The number of dying neurons in the central body between the level of the ellipsoid body and the giant fiber commissure was counted 10 days as described²³. At least six hemibrains were analyzed for each genotype. Expression was confirmed in EP lines by in situ hybridization to third instar larval CNS preparations with the EP element of interest transheterozygous to GMR-GAL4 following a standard protocol.⁹¹ For electron microscopy, adult flies were dehydrated through a graded series of ethanol solutions, critical point dried, sputter coated, and examined with a scanning electron microscope.

III. Results

A Genetic Screen for Modifiers of Tau Toxicity

The Drosophila tauopathy model is based on the GAL4-UAS expression system, in which a human tau transgene downstream of a yeast upstream activating sequence (UAS) is controlled by driver lines which express the GAL4 transcriptional activator in particular spatial and temporal patterns.^{23,24} When Tau expression is targeted to the Drosophila eye using the GMR-GAL4 driver line, adult flies show a rough eye phenotype. In comparison to the normal fly eye, expression of human Tau reduces the size of the eye and disrupts the regular array of lenses, reflecting disorganization of the underlying photoreceptor clusters, termed ommatidia. The severity of the rough eye phenotype correlates well with the level of Tau expression, suggesting that the phenotype should be a sensitive substrate for second site genetic modification. Toward this end, we chose a genotype, UAS-tauv³³⁷/+; GMR-GAL41+, with a moderately rough eye, allowing us to identify both enhancers and suppressors of Tau toxicity. We screened insertion lines containing EP transposable elements.²⁵ When the EP element is inserted proximal to a gene, and in the same orientation, it allows the ectopic expression of the locus under the control of GAL4. Alternatively, when inserted in the reverse orientation, the EP element often inactivates expression of the gene. Thus, our EP screen identifies both gain-of-function and loss-of-function modifiers of Tau toxicity.

We carried out an F1 screen of an established collection of 2,276 EP transposable elements by crossing flies expressing human Tau in the eye to individual EP insertion lines and examining the progeny for dominant enhancement or suppression of the Tau-induced rough eye phenotype.²⁶ Suppressors of Tau toxicity in the eye restored the eye to normal size, and significantly ameliorated the ommatidial irregularity. In contrast, enhancers of the Tau rough eye phenotype further reduced the eye in size and produced increased ommatidial irregularity and fusion of the overlying lenses. The screening of Tau modifiers was carried out in a blinded, unbiased fashion, without knowledge of the insertion positions or identity of the affected genes.

All candidate modifiers were subjected to a series of validation tests. We first generated precise excisions for each EP line to demonstrate reversion of the modifier activity, and only pursued those EP lines that showed significant enhancement or suppression of Tau toxicity relative to background chromosome effects. Next, all of the candidate enhancers were test-crossed to GMR-GAL4. We discarded any lines that caused a moderate or severe rough eye phenotype on their own. We did retain a limited subgroup of modifiers (EP(2)2028, EP(2)2437, EP(3)3517, and EP(3)3559) that produced a very mild rough eye in combination with GMR-GAL4. However, expressing Tau in combination with these EP elements produced a severe rough eye, consistent with synergistic enhancement by the EP elements.

The EP insertion position, orientation, and candidate loci were determined using the on-line resources of the Berkeley Drosophila Genome Project and FlyBase.^{27,28} Most of the modifier genes had EP insertions in the corresponding transcription units. In a few cases, the EP element was inserted within 5 kb proximal to the transcriptional start site. In several instances, multiple insertions were recovered affecting the same locus. In one notable case, the insertions EP(3)3569 and EP(3)1072 were independently recovered as a suppressor and enhancer, respectively, and were inserted at the same genomic position but in opposite orientations demonstrating both gain of function and loss of function effects. Where the EP element was inserted proximal to and in the same orientation as a candidate gene (22/29 cases), we could often validate overexpression of the predicted locus. For many loci, previously published UAS transgenic stocks were obtained and tested for modifier activity. In several other cases, we performed in situ hybridization to demonstrate enhanced expression of the locus under the control of GMR-GAL4. Finally, where possible, we tested mutant alleles of the candidate loci as Tau modifiers. In two cases, analysis of mutant alleles revealed that gain-of-function and loss-of-function of the same locus modified Tau neurotoxicity in opposite directions. Finally, we performed Western blot analysis on all candidate suppressors and demonstrated that none of the suppressors simply reduced Tau expression. The resulting suppressors and enhancers of Tau toxicity that fulfilled all validation criteria are presented in Table 3 (see pages 24 and 25 below). Table 3 also shows the results of the validation tests for each modifier.

In addition to retinal toxicity, expression of human Tau in flies produces age dependent degeneration of neurons in the brain.²³ To determine the effect of our modifiers in the brain, we expressed human Tau in a panneural pattern using the elav-GAL4 driver. We then compared the number of dying neurons present in the brains of aged Tau transgenic flies in the presence or absence of genetic modifiers. Enhancers of retinal toxicity also enhanced neurodegeneration in the brain and suppressors of retinal toxicity also suppressed neuronal loss in the brain. The loss of function enhancer EP(3)1072 did not cause neurodegeneration in the absence of Tau expression. Nor did crossing the gain of function enhancer EP(3)3319 to elav-GAL4 induce cell death in the brain in the absence of the Tau transgene. Thus, the elevated levels of neuronal death observed with these enhancers was specific to Tau-induced neurodegeneration.

Kinases and Phosphatases are the Major Class of Tau Modifiers

The largest functional class of modifiers encodes kinases or phosphatases, including Drosophila homologs of several enzymes known to phosphorylate or dephosphorylate Tau (Table 3, pages 24 and 25 below). EP(2)0899, a Tau suppressor, is predicted to activate expression of the fly ortholog of the MARK/PAR-1 serine/threonine kinase. MARK was initially isolated biochemically based on its ability to phosphorylate Tau at Ser262, and antibodies raised against this phosphoepitope show relative specificity for Tau isolated from Alzheimer's disease brain.^{29,30} Suppression of the Tau rough eye phenotype by increasing PAR-1 expression was confirmed using a UAS-par1 transgene.

We also identified subunits of the PP1 and PP2A phosphatases as modifiers. Both of these enzymes bind Tau and can dephosphorylate the protein in vitro.³¹⁻³⁴ EP(3)3518 was identified as a suppressor (Table 3), and is predicted to overexpress a regulatory subunit of PP1. We confirmed overexpression by mRNA in situ hybridization. EP(3)3559, previously shown to activate expression of a PP2A regulatory subunit,35 was identified as a Tau enhancer. Since PP1 and PP2A regulatory subunits can either activate or inhibit catalytic activity, depending on the substrate, it is difficult to predict how expression of the subunits we have identified might effect phosphorylation of Tau or other potential substrates.³⁶ Nevertheless, recovery of known Tau kinases and phosphatases in an unbiased genetic screen provides an in vivo validation of their importance in tauopathy pathogenesis.

In addition to MARK/PAR-1, three additional serine/threonine kinases were recovered in our screen. All of these proteins have well-conserved mammalian homologs. EP(3)3319, a Tau enhancer, is predicted to activate expression of the Center divider kinase.³⁷ Another enhancer of Tau toxicity, EP(X)1455, activates expression of CG14217, a Drosophila homolog of the STE20-related kinase, Tao_{1.}³⁸ EP(3)1246, a Tau suppressor, is a potential inactivating insertion in the first intron of CG10967, which encodes an ortholog of the C. elegans Unc-51 kinase.³⁹ Our screen also identified two Drosophila homologs of the CDC25 phosphatase, string and twine, as suppressors of Tau. Three activating insertions in string, EP(2)1213, EP(2)3426, and EP(2)3432, were recovered independently as Tau suppressors. We confirmed the ability of String to suppress Tau toxicity using a UAS-string transgene. Twine was identified as a single activating insertion, EP(2)613. The recovery of String and Twine as Tau modifiers in our genetic screen is intriguing given that the expression and activity of Cdc25 and its substrate, Cdc2, have both been found to be dysregulated in the Alzheimer's brain.⁴⁰⁻⁴²

Genetic Modifiers Implicate Apoptosis in Tau Toxicity

In addition to kinases and phosphatases, we have identified a number of other genetic modifiers that help elucidate the mechanism of Tau toxicity. Two of our enhancers have been implicated in apoptotic regulation. Thread (Th), a Drosophila homolog of the inhibitor of apoptosis proteins (IAPs), binds and inactivate pro-apoptotic caspases.^{43,44} EP(3)3308, a Tau suppressor, is predicted to activate expression of th. We have confirmed that overexpression of Th suppresses Tau toxicity using UAS-th and GMR-th transgenes. Reciprocally, a threadloss-of-function allele, th^{iC58}, and a dominant negative allele, th^{SL}, both enhanced the tau rough eye. The other apoptosis-related modifier that we identified, EP(2)2504, is predicted to express a homolog of the C. elegans Fem-1 protein.⁴⁵ Fem-1 is a substrate for the apoptotic caspase, CED-3, binds directly to the apoptotic regulator CED-4, and induces apoptosis in cultured cells.⁴⁶ A putative role for Fem-I in apoptosis is likely conserved across species, since a mammalian homologue, F1α, directly interacts with the cytoplasmic domains of Fas and the tumor necrosis factor receptor 1, and induces apoptosis in mammalian cells.⁴⁷ The role of apoptosis in the Alzheimer's disease and related disorders, remains controversial,⁴⁸ however, our results suggest that Tau-induced cell death in flies, and therefore in human disease, may be apoptotic.

Novel Mediators of Tau Toxicity

Two of our modifiers, EP(3)3145 and EP(3)3517, alter the expression of Drosophila homologs of genes mutated in human neurological diseases (Table 3, pages 24 and 25 below). EP(3)3145 increases the expression of an Ataxin-2 homolog. In patients with spinocerebellar ataxia type 2, mutant Ataxin-2 contains a polyglutamine repeat expansion and induces degeneration of cerebellar Purkinje cells, brainstem nuclei, and spinal cord nuclei and tracts.⁴⁹⁻⁵² EP(3)3517 activates expression of the Drosophila homolog of the Fragile-X mental retardation protein (Fmr1). An inactivating trinucleotide repeat expansion in human FMRP causes the most common inherited form of mental retardation.⁵³ In flies, Fmr1 represses translation of the microtubule associated protein Futsch, a Drosophila Map1b homolog.⁵⁴ Our screen also identified a second protein implicated in microtubule function. Expression of the Drosophila microtubule associated protein Orbit^{55,56} via the EP(3)3403 element enhanced Tau toxicity. In addition, we have identified cheerio, a Drosophila ortholog of the actin binding protein, Filamin, as a Tau enhancer.⁵⁷ The relationship between the normal function of Tau as a microtubule associated protein and its pathological role in human disease remains unknown; however, our identification of Orbit, Fmr1, and Cheerio as enhancers of Tau neurotoxicity suggests that the neuronal actin and microtubule cytoskeletons may play an important role in tauopathy pathogenesis.

Lastly, our screen has identified several novel, conserved genes that may define important biochemical steps mediating Tau toxicity. This modifier class includes the suppressors, EP(2)2090, EP(2)2311, and EP(2)2475, as well as the enhancers, EP(2)2510, EP(2)2190, EP(3)326, and EP(2)2437. Future studies of these genes in Drosophila should delineate the biological functions of these novel proteins. Additional investigation in Alzheimer's disease brains and in vertebrate models of Alzheimer's disease and related disorders⁵⁸ will be required to define their role in disease pathogenesis.

Known Tau Kinases Modulate Tau Toxicity In Vivo

Given the number of kinases and phosphatases identified by our screen, we determined if other kinases known to phosphorylate Tau in vitro could modify Tau toxicity in vivo. Members of the MARK superfamily phosphorylate Tau in an terminal proline-rich domain. In particular, the c-jun-terminal kinase (JNK) and stress-activated protein kinase subfamily has been implicated in pathological Tau phosphorylation, and the activity of this pathway is abnormally upregulated in Alzheimer's brain.⁵⁹⁻⁶² Expression of Hemipterous, the Drosophila homolog of JNK-kinase, activates the JNK pathway in the Drosophila eye⁶³ and enhances Tau toxicity. Co-expression of Hemipterous with Tau produced a decrease in eye size, increased surface roughness, and induced the formation of necrotic black patches, as compared with control flies expressing Tau alone. Expression of Hemipterous alone in the eye under the control of GMR-GAL4 did not affect eye morphology.

Like the MARK kinase, Protein kinase A (PKA) can phosphorylate residues within the Tau microtubule binding repeats (Ser262, Ser324, and Ser356), and can additionally mediate phosphorylation within a flanking domain at Ser214.^{64,65} To test the effect of PKA, we used a constitutively active version of murine PKA that had no effect on the Drosophila eye when expressed alone. Expression of active mouse PKA enhanced the rough eye phenotype produced by human Tau. In addition, a constitutively active version of Drosophila PKA strongly enhanced the rough eye caused by expressing human Tau in photoreceptor cells of the retina using elav-GAL4.

The Cdc2-related kinase, Cdk5, has received significant attention as a potential mediator of Tau phosphorylation in disease. The Cdk5 regulatory subunit, p35, is abnormally cleaved to p25 in Alzheimer's brain, and the resulting p25/Cdk5 complex has enhanced Tau kinase activity.¹⁷ When tested individually, neither Cdk5 nor p35 expression modified the Tau rough eye phenotype. However, co-expression of both Cdk5 and p35 potently enhanced the rough eye produced by Tau. Although expressing both Cdk5 and p35 in the eye using GMR-GAL4 driver produced a mild rough eye, co-expression of Cdk5/p35 with human Tau resulted in synergistic retinal toxicity including large necrotic patches and sunken areas representing loss of underlying retinal tissue. In addition, when crosses were carried out at 18° C. to decrease the activity of the GAL4-UAS expression system, Cdk5/p35 expression no longer caused a rough eye alone, but still markedly enhanced the Tau rough eye phenotype.

Glycogen synthase kinase 313 (GSK3β) phosphorylates Tau in vitro and participates in the formation of disease-associated phosphoepitopes.^{65,68} Tau is also hyperphosphorylated in vivo following neuronal GSK3β expression in transgenic mice.⁶⁹ When flies were raised at 18° C., neither expression of human Tau nor expression of GSK3β affected the morphology of the eye. However, expression of both transgenes together produced an obvious rough eye phenotype.

We have demonstrated that activation of the JNK pathway, PKA, GSK3β, and Cdk5 each enhance Tau toxicity in vivo, in addition to the well established ability of these kinases to phosphorylate Tau in vitro. Together with the modifiers identified by our unbiased genetic screen, these results demonstrate that kinases and phosphatases control Tau toxicity in vivo, and support a link between Tau phosphorylation and neurodegeneration in human disorders, like Alzheimer's disease, characterized by extensive Tau pathology.

A Subgroup of Tau Modifiers Show Genotype-Specific Activity

Although mutations in the tau gene cause rare familial tauopathies, the vast majority of neurodegenerative disease associated with Tau pathology involves the abnormal phosphorylation and deposition of wild-type Tau. The relationship between the toxicity of wild-type Tau in Alzheimer's disease and that of mutant Tau in FTDP-17 is unclear. We have previously shown that expression of either wild-type or mutant human tau transgenes causes neurodegeneration in Drosophila, although mutant Tau is more toxic than wild-type Tau.²³ Our genetic screen was performed using Tau^{V337M}. To investigate the relationship between the toxicity of wild-type and mutant Tau, we tested the ability of each of our modifiers to alter the toxicity of Tau WT, as well as the toxicity of a second disease-linked mutant Tau protein, Tau.^{R406W}

Nearly all of the modifiers isolated for their ability to alter the toxicity of TauV^{337M} showed consistent effects across all three Tau genotypes, suggesting that they define genetic pathways mediating the toxicity of wild-type and mutant forms of Tau alike. However, we have also identified a small subgroup of modifiers that showed genotype-specific effects. One suppressor, EP(2)0418, and two enhancers, EP(3)3319 and EP(3)1051, selectively modified either TauV^{337M} and Tau^{R406W}, but had no effect on the toxicity of Tau.^{WT}

Since our screen was conducted with mutant Tau, we could not identify modifiers with a completely specific effect on wild-type Tau; however, one enhancer, EP(3)3659, did function as a more potent enhancer of wild-type Tau when compared to its activity on mutant Tau proteins. In sum, while most of our genetic modifiers have conserved effects, a subset define distinct pathways responsible for the differences in toxicity of wild-type versus mutant Tau.

Most Tau modifiers do not Affect Polyglutamine Toxicity

Although neurodegenerative disorders like Alzheimer's disease, Parkinson's disease, and Huntington's disease have distinct clinical manifestations, they all involve adult onset of disease, loss of specific neuronal subpopulations, and the formation of abnormal protein aggregates. In addition, familial forms of these disorders are inherited in a dominant fashion, consistent with a toxic gain-of-function mechanism. Such parallels have led to speculation that these diseases might share fundamentally similar mechanisms of pathogenesis, perhaps related to abnormalities of protein aggregation and degradation.²² However, this hypothesis has been difficult to test directly. We have now used Drosophila genetics to investigate the relationship between the toxicity of Tau and polyglutamine repeat containing proteins.

We first tested the activity of all of our Tau modifiers in a polyglutamine disease model, spinocerebellar ataxia type 1 (SCA 1). Expression of a human SCA1 transgene with an expanded polyglutamine track produces a moderately rough and depigmented eye.^{18,70} 27 of 29 Tau modifiers had no effect on the eye phenotype produced by expression of mutant human SCA1. In particular, all of the kinases and phosphatases that potently affect Tau toxicity failed to modify the SCA1-induced eye phenotype, suggesting that this functional group is not a determinant of polyglutamine toxicity. Tau modifiers did enhance SCA1 toxicity, EP(2)2510 and EP(3)3145. Both modifiers enhanced the loss of eye pigmentation and induced necrotic black dots. Interestingly, EP(3)3145 appears to activate expression of a Drosophila homolog of Ataxin-2. Expansion of a polyglutamine tract in human Ataxin-2 produces a spinocerebellar ataxia with clinical and neuropathological similarities to SCA 1.

In contrast to the results of our Tau modifier screen, previous Drosophila genetic screens for modifiers of polyglutamine toxicity have not identified kinases and phosphatases. A number of studies have instead implicated heat shock proteins, chaperones, and components of the ubiquitin-proteasome pathway as key determinants of polyglutamine toxicity. 18-20 These results suggest that abnormalities in protein folding, aggregation, and degradation underlie neurotoxicity in polyglutamine repeat diseases. In contrast, none of the Tau modifiers identified in our forward genetic screen directly control protein folding or degradation, suggesting a distinct mechanism of toxicity. We have further examined the relationship between Tau and polyglutamine toxicity by directly testing all of the previously identified modifiers of polyglutamine toxicity in our tauopathy model. Consistent with the results above, nearly all of these modifiers, including numerous heat shock proteins, chaperones, and ubiquitin pathway components, fail to modify Tau toxicity. These results fit well with our previous finding that neurodegeneration in the Drosophila tauopathy model correlates with the formation of abnormal Tau phosphoepitopes, but not with the large Tau aggregates known as neurofibrillary tangles.²³

TABLE 3
Suppressors and Enhancers of Tau

| | | | | EP Orientation/ | Allelles tested/ |
|---|---|---|---|---|---|
| Line | Modification¹ | Gene | Mammalian homolog/function | overexpression² | modification |
| EP(2)0899³ | Su | par-1 | MARK serine/threonine kinase | S/U | par-1W3/none |
| EP(3)1246⁴ | Su | CG10967 | Unc-51 serine/threonine kinase | O | n.a. |
| EP(X)1455 | En | CG14217 | Tao1 serine/threonine kinase | S | n.a. |
| EP(3)3319 | En | center divider | TESK1 serine/threonine kinase | S | cdi07013/none |
| EP(2)1213³ | Su | string | CDC25 phosphatase | S/U | stg01235/none |
| EP(2)0613 | Su | twine | CDC25 phosphatase | S | twek08310/none |
| EP(3)3518⁴ | Su | CG9238 | PP1A phosphatase subunit | S | ** |
| EP(3)3559 | En | CG5643 | PP2A phosphatase subunit | S/I | n.a. |
| EP(3)3569³ | Su | CG13610 | organic cation transporter | S | n.a. |
| EP(3)1072 | En | CG13610 | organic cation transporter | O | n.a. |
| EP(3)3308 | Su | thread | IAP1 apoptosis inhibitor | S/U | thj5C8/En; thSL/En |
| EP(2)2504 | En | CG9025 | Fem1 apoptosis activator | O | n.a. |
| EP(2)2208 | Su | wunen | phosphatidic acid phosphatase | S/U | Df(2R)w73-1/En |
| EP(2)0712 | Su | CG10082 | inositol hexokisphosphate kinase | O | n.a. |
| EP(2)3403 | En | orbit/mast | microtubule associated protein | S | not tested |
| EP(3)3145 | En | CG5166 | Ataxin2 | S | 1(3)06490/None |
| EP(3)3517 | En | CG6203 | Fragile-X | S | not tested |
| EP(3)3265 | En | buttonless | transcription factor | S | not tested |
| EP(2)2500 | En | yan/aop | transcriptional repressor | S/U | aop1/none |
| EP(3)0581³ | En | dally | glypican | S/U | dlyP16852A/none |
| EP(2)2028 | En | CG9897 | sec7GEF | S/I | n.a. |
| EP(2)2090 | Su | CG5859 | novel | S | n.a. |
| EP(2)2311 | Su | CG33735 | novel | S | n.a. |
| EP(2)2475 | Su | CG11166 | novel | S | n.a. |
| EP(2)2510 | En | CG7231 | novel | O | n.a. |
| EP(2)2190 | En | CG10927 | novel | O | n.a. |
| EP(3)0326 | En | furry | novel | S | not tested |
| EP(2)2437³ | En | SD02913 | novel | O | n.a. |

¹En, enhancer; Su, suppressor.
²Orientation of EP element relative to transcription unit, and validation of overexpression, if applicable. S, same; O, opposite; U, UAS-transgene; I, mRNA in situ hydbridization.
³Multiple independent insertions were identified which gave similar modification, but only one is listed, for simplicity.
⁴Lines for which incomplete reversion was observed.

IV. Discussion

Multiple lines of evidence support a central role for Tau in the pathogenesis of Alzheimer's and related neurodegenerative diseases. Most significantly, neurofibrillary tangle pathology correlates well with neuronal loss and cognitive dysfunction, and mutations in the tau gene cause the neurodegenerative syndrome, FTDP-17.¹⁻⁵ Here we report a genetic dissection of the mechanisms responsible for Tau neurotoxicity. The application of Drosophila genetics to the study of neurodegenerative disease permits an unbiased survey of the relevant molecular mechanisms of disease pathogenesis.

Our screen has identified 17 enhancers and 12 suppressors of Tau toxicity. Nearly a third of these modifiers encode protein kinases and phosphatases, the largest single functional class we recovered. Remarkably, several of these modifiers, including the MARK kinase and the PP 1 and PP2A phosphatases, have been previously shown to phosphorylate or dephosphorylate Tau in vitro. The recovery of these kinases and phosphatases by our unbiased genetic screen provides an in vivo validation of their importance for disease pathogenesis. We further demonstrate that several candidate kinases, including Cdk5, GSK3β, PKA, and the JNK pathway, that are known to phosphorylate Tau in vitro, also enhance Tau toxicity in vivo.

Many of the kinases and phosphatases that control Tau neurotoxicity in transgenic flies have been previously implicated in the pathogenesis of Alzheimer's disease based on alterations in localization or activity in postmortem brain samples from patients. The MARK kinase and activated JNK colocalize tightly with neurofibrillary tangles.^{61,62,71} PP2A mRNA levels are abnormally decreased in Alzheimer's disease brains.⁷² Similarly, the expression and activity of Cdc25 and its substrate, Cdc2, have both been found to be dysregulated in Alzheimer's brain.^{40,42} The Cdk5 regulatory subunit, p35, can be abnormally cleaved to p25 in Alzheimer's brain, resulting in constitutive activity of Cdk5.⁶⁷ Furthermore, the Amyloid-13 peptide can induce p35 cleavage via activation of the protease Calpain, raising the possibility that Cdk5 may serve as a pathological link between the amyloid and Tau lesions of Alzheimer's⁷³. Our finding that these kinases and phosphatases, which have altered distributions and/or activities in disease states, can also control Tau toxicity in vivo supports the identification of these enzymes as key therapeutic targets in Alzheimer's disease and related disorders.

We have also identified several additional conserved serine/threonine kinases as Tau modifiers. Activating expression of the Center divider kinase and a Drosophila homolog of the Tao1 kinase enhanced Tau toxicity, while a putative inactivating insertion in the rosophila ortholog of the Unc-51 kinase suppressed Tau toxicity. The Center divider kinase is expressed in the developing Drosophila nervous system and has a well conserved mammalian homolog.³⁷ Tao1 is highly expressed in the rat brain.³⁸ Unc-51 plays a critical role in neuronal process formation in the nematode and in cerebellar granule cells in the mouse.^{39,74} Thus, we have identified three conserved, neuronally-expressed serine/threonine kinases that can control Tau toxicity.

Tau isolated from the brains of patients dying with Alzheimer's disease and other tauopathies is abnormally hyperphosphorylated, and many Tau phosphoepitopes are specifically associated with disease in the adult brain. These observations have long fueled speculation that phosphorylation of Tau determines neurotoxicity. However, direct experimental demonstration that phosphorylation controls neurodegenerative cell death in vivo has been difficult. We have previously shown that, as in human disease, transgenic human Tau is abnormally phosphorylated in the Drosophila brain and that the development of disease-linked Tau phosphoepitopes correlates both spatially and temporally with neuronal degeneration.²³ Our finding that kinases and phosphatases are the major determinants of neurodegeneration in our Drosophila model, including several enzymes known to directly phosphorylate or dephosphorylate Tau, strongly supports a link between Tau phosphorylation and neurotoxicity in vivo.

A number of in vitro studies have demonstrated that hyperphosphorylation decreases the affinity of Tau for microtubules and increases homotypic interactions, thus potentially favoring cytosolic accumulation and aggregation in vivo.^{13,14} Many of the mutations in tau that cause FTDP-17 similarly reduce the interaction of Tau with microtubules and promote Tau oligomerization.^{1,15} In striking concordance with human disease, phosphorylation and disease-linked mutations are both major correlates of enhanced neurotoxicity in our Drosophila tauopathy model²³. Thus, decreased microtubule affinity, increased aggregation, or both may enhance the neurotoxicity of Tau in flies. Interestingly, the one exception to our finding that increasing kinase expression correlates with enhanced Tau toxicity is MARK/PAR-1 which behaves as a genetic suppressor and is known to phosphorylate Tau at Ser262²⁹. While phosphorylation of Ser262 within the microtubule binding domain abolishes the binding of Tau to microtubules in vitro and in vivo, phosphorylation at this site was also found to strongly inhibit aggregation⁶⁴. Although significant numbers of large filamentous Tau aggregates are not present in flies expressing human tau,²³ our identification of MARKIPAR-1 as a suppressor may be consistent with the possibility of a smaller, perhaps protofilamentous, toxic aggregate.

A number of neurodegenerative diseases have now been successfully modeled in Drosophila¹⁷, including Parkinson's disease⁷⁰, tauopathies²³, Huntington's disease⁷⁵, and two of the spinocerebellar ataxias^{18,76}. These and related models have been used to identify genetic modifiers.^{18-21,77} The availability of multiple Drosophila models of neurodegenerative diseases, and a growing collection of genetic modifiers, allows us to compare cellular pathways controlling neurodegenerative cell death. Similarities in the clinical and neuropathologic features of the cognate human neurodegenerative diseases have suggested that the disorders may share similar mechanisms of pathogenesis²².

In contrast, we present evidence supporting distinct mechanisms of toxicity in polyglutamine disorders and tauopathies. First, our Tau screen identified a completely non-overlapping group of modifiers compared with previous screens for polyglutamine modifiers.

In at least one case, the identical collection of EP-elements was screened¹⁸. The largest class of polyglutamine modifiers recovered to date consists of chaperones and ubiquitin-proteasome pathway components. We have not identified any of these genes in our screen. Instead, the largest single class of Tau modifiers includes kinases and phosphatases. Second, we have tested all of our Tau modifiers in a Drosophila SCA1 model. Most show no effect on polyglutamine toxicity. Third, we have also tested most of the published modifiers of polyglutamine toxicity in our tauopathy model, and found that nearly all have no effect on Tau toxicity. These results suggest that Tau and polyglutamine toxicities in Drosophila are mostly controlled by distinct sets of genes with roles in different biological processes. Thus, diverse therapeutic approaches may be required in neurodegenerative diseases that seemingly share key similarities.

Although the majority of Tau and polyglutamine modifiers define non-overlapping sets, we did identify a few exceptions. Two EP enhancers from our Tau screen also enhanced SCA1.

One encodes a novel protein, and the other activates expression of a Drosophila Ataxin-2 homolog. These modifiers may define convergent pathways of toxicity. In addition, the chaperone DnaJ-1 suppresses both Tau toxicity and polyglutamine toxicity.¹⁹ The ability of DnaJ-1, but not other molecular chaperones to influence Tau toxicity may correlate with the specific association between abnormal conformational epitopes of Tau and neurodegeneration in Alzheimer's and related diseases. The most specific immunohisto-chemical markers of neurodegeneration in the brains of transgenic flies expressing mutant human Tau are the antibodies Alz-50 and MC1.²³ These antibodies recognize abnormal conformational variants of Tau specific to disease state¹¹. The Alz-50 epitope is determined by both—and C-terminal Tau sequences, but not intervening residues, indicating abnormal folding of the protein. DnaJ-I may specifically inhibit the abnormal folding of Tau and thus prevent neurotoxicity.

In conclusion, an analysis of modifiers recovered in our screen suggests a genetic pathway for Tau toxicity in human disease. We propose that kinases and phosphatases play a critical early role in disease pathogenesis, perhaps by modulating the affinity of Tau for microtubules and thereby increasing the cytoplasmic Tau fraction. Elevated levels of free Tau favor the formation of an abnormally folded, toxic Tau species. The next step in this cascade remains a key biological mystery; however, our genetic modifiers may identify some of the relevant molecular pathways. Expression of the Drosophila homologs of two proteins implicated in distinct human neurologic diseases, Ataxin-2 and the Fragile X mental retardation protein, enhances Tau neurotoxicity, raising the possibility of unexpected mechanistic similarities among these disorders.

In addition, our screen has identified several novel, highly conserved proteins that may transduce the toxic effects of abnormal Tau. Our recovery of multiple modifiers which function in cytoskeletal regulation may implicate the neuronal cytoskeleton as a possible subcellular target. Finally, our finding that genetic modifiers related to apoptosis also influence Tau neurotoxicity, provides additional experimental evidence that apoptosis is the end pathway of neurodegenerative cell death in Alzheimer's disease and tauopathies⁴⁸. This genetic scheme of Tau-induced neurodegeneration highlights numerous targets for possible therapeutic intervention. In addition, determinants of Tau toxicity in Drosophila identify candidate loci for familial neurodegenerative syndromes as well as potential modifier genes in Alzheimer's disease and related disorders.

### REFERENCES

1. Hong, M. et al. Mutation-specific functional impairments in distinct tau isoforms of hereditary FTDP-17. Science 282, 1914-7. (1998).

2. Hutton, M. et al. Association of missense and 5′-splice-site mutations in tau with the inherited dementia FTDP-17. Nature 393, 702-5. (1998).

3. Spillantini, M. G. et al. Mutation in the tau gene in familial multiple system tauopathy with presenile dementia. Proc Natl Acad Sci USA 95, 7737-41. (1998).

4. Arriagada, P. V., Growdon, J. H., Hedley-Whyte, E. T. & Hyman, B. T. Neurofibrillary tangles but not senile plaques parallel duration and severity of Alzheimer's disease. Neurology 42, 631-9. (1992).

5. Braak, H. & Braak, E. Neuropathological stageing of Alzheimer-related changes. Acta Neuropathol 82, 239-59 (1991).

6. Lee, V. M., Goedert, M. & Trojanowski, J. Q. Neurodegenerative tauopathies. Annu Rev Neurosci 24, 1121-59 (2001).

7. Grundke-Iqbal, I. et al. Abnormal phosphorylation of the microtubule-associated protein tau (tau) in Alzheimer cytoskeletal pathology. Proc Natl Acad Sci USA 83, 4913-7. (1986).

8. Ihara, Y., Nukina, N., Miura, R. & Ogawara, M. Phosphorylated tau protein is integrated into paired helical filaments in Alzheimer's disease. J Biochem (Tokyo) 99, 1807-10. (1986).

9. Lee, V. M., Balin, B. J., Otvos, L., Jr. & Trojanowski, J. Q. A68: a major subunit of paired helical filaments and derivatized forms of normal Tau. Science 251, 675-8. (1991).

10. Hasegawa, M. et al. Characterization of mAb AP422, a novel phosphorylation-dependent monoclonal antibody against tau protein. FEBS Lett 384, 25-30. (1996).

11. Jicha, G. A. et al. A conformation- and phosphorylation-dependent antibody recognizing the paired helical filaments of Alzheimer's disease. J Neurochem 69, 2087-95. (1997).

12. Matsuo, E. S. et al. Biopsy-derived adult human brain tau is phosphorylated at many of the same sites as Alzheimer's disease paired helical filament tau. Neuron 13, 989-1002. (1994).

13. Alonso, A., Zaidi, T., Novak, M., Grundke-Iqbal, I. & Iqbal, K. Hyperphosphorylation induces self-assembly of tau into tangles of paired helical filaments/straight filaments. Proc Natl Acad Sci USA 98,6923-8. (2001).

14. Gustke, N. et al. The Alzheimer-like phosphorylation of tau protein reduces microtubule binding and involves Ser-Pro and Thr-Pro motifs. FEBS Lett 307, 199-205. (1992).

15. Hasegawa, M., Smith, M. J. & Goedert, M. Tau proteins with FTDP-17 mutations have a reduced ability to promote microtubule assembly. FEBS Lett 437, 207-10. (1998).

16. Lovestone, S. & Reynolds, C. H. The phosphorylation of tau: a critical stage in neurodevelopment and neurodegenerative processes. Neuroscience 78, 309-24. (1997).

17. Muqit, M. M. K. & Feany, M. B. Modelling neurodegenerative diseases in Drosophila: a fruitful approach? Nat Rev Neurosci 3, 237-243 (2002).

18. Fernandez-Funez, P. et al. Identification of genes that modify ataxin-1-induced neurodegeneration. Nature 408, 101-6. (2000).

19. Kazemi-Esfarjani, P. & Benzer, S. Genetic suppression of polyglutamine toxicity in Drosophila. Science 287, 1837-40. (2000).

20. Warrick, J. M. et al. Suppression of polyglutamine-mediated neurodegeneration in Drosophila by the molecular chaperone HSP70. Nat Genet 23, 425-8. (1999).

21. Auluck, P. K., Chan, H. Y., Trojanowski, J. Q., Lee, V. M. & Bonini, N. M. Chaperone suppression of alpha-synuclein toxicity in a Drosophila model for Parkinson's disease. Science 295, 865-8. (2002).

22. Trojanowski, J. Q. & Lee, V. M. “Fatal attractions” of proteins. A comprehensive hypothetical mechanism underlying Alzheimer's disease and other neurodegenerative disorders. Ann NY Acad Sci 924, 62-7 (2000).

23. Wittmann, C. W. et al. Tauopathy in Drosophila: neurodegeneration without neurofibrillary tangles. Science 293, 711-4. (2001).

24. Brand, A. H. & Perrimon, N. Targeted gene expression as a means of altering cell fates and generating dominant phenotypes. Development 118, 401-415 (1993).

25. Rorth, P. A modular misexpression screen in Drosophila detecting tissue-specific phenotypes. Proc Natl Acad Sci USA 93, 12418-22. (1996).

26. Rorth, P. et al. Systematic gain-of-function genetics in Drosophila. Development 125, 1049-57. (1998).

27. Adams, M. D. et al. The genome sequence of Drosophila melanogaster. Science 287, 2185-2195 (2000).

28. FlyBase. The FlyBase database of the Drosophila Genome Projects and community literature. Nucleic Acids Res 27, 85-8. (1999).

29. Drewes, G., Ebneth, A., Preuss, U., Mandelkow, E. M. & Mandelkow, E. MARK, a novel family of protein kinases that phosphorylate microtubule-associated proteins and trigger microtubule disruption. Cell 89, 297-308 (1997).

30. Hasegawa, M. et al. Protein sequence and mass spectrometric analyses of tau in the Alzheimer's disease brain. J Biol Chem 267, 17047-17054 (1992).

31. Goedert, M., Jakes, R., Qi, Z., Wang, J. H. & Cohen, P. Protein phosphatase 2A is the major enzyme in brain that dephosphorylates tau protein phosphorylated by proline-directed protein kinases or cyclic AMP-dependent protein kinase. J Neurochem 65, 2804-7. (1995).

32. Liao, H., Li, Y., Brautigan, D. L. & Gundersen, G. G. Protein phosphatase 1 is targeted to microtubules by the microtubule-associated protein Tau. J Biol Chem 273, 21901-8. (1998).

33. Sontag, E. et al. Molecular interactions among protein phosphatase 2A, tau, and microtubules. Implications for the regulation of tau phosphorylation and the development of tauopathies. J Biol Chem 274, 25490-8. (1999).

34. Yamamoto, H., Saitoh, Y., Fukunaga, K., Nishimura, H. & Miyamoto, E. Dephosphorylation of microtubule proteins by brain protein phosphatases 1 and 2A, and its effect on microtubule assembly. J Neurochem 50, 1614-23. (1988).

35. Kraut, R., Menon, K. & Zinn, K. A gain-of-function screen for genes controlling motor axon guidance and synaptogenesis in Drosophila. Curr Biol 11, 417-30. (2001).

36. Bollen, M. Combinatorial control of protein phosphatase-1. Trends Biochem Sci 26,426-31. (2001).

37. Matthews, B. B. & Crews, S. T. Drosophila center divider gene is expressed in CNS midline cells and encodes a developmentally regulated protein kinase orthologous to human TESK1. DNA Cell Biol 18, 435-48. (1999).

38. Hutchison, M., Berman, K. S. & Cobb, M. H. Isolation of TAO1, a protein kinase that activates MEKs in stress-activated protein kinase cascades. J Biol Chem 273,28625-32. (1998).

39. Ogura, K. et al. Caenorhabditis elegans unc-51 gene required for axonal elongation encodes a novel serine/threonine kinase. Genes Dev 8, 2389-400. (1994).

40. Ding, X. L. et al. The cell cycle Cdc25A tyrosine phosphatase is activated in degenerating postmitotic neurons in Alzheimer's disease. Am J Pathol 157, 1983-90. (2000).

41. Vincent, I., Jicha, G., Rosado, M. & Dickson, D. W. Aberrant expression of mitotic cdc2/cyclin B 1 kinase in degenerating neurons of Alzheimer's disease brain. J Neurosci 17, 3588-98. (1997).

42. Vincent, I. et al. Constitutive Cdc25B tyrosine phosphatase activity in adult brain neurons with M phase-type alterations in Alzheimer's disease. Neuroscience 105,639-50 (2001).

43. Hay, B. A., Wassarman, D. A. & Rubin, G. M. Drosophila homologs of baculovirus inhibitor of apoptosis proteins function to block cell death. Cell 83, 1253-62. (1995).

44. Lisi, S., Mazzon, I. & White, K. Diverse domains of THREAD/DIAPI are required to inhibit apoptosis induced by REAPER and HID in Drosophila. Genetics 154, 669-78. (2000).

45. Doniach, T. & Hodgkin, J. A sex-determining gene, fem-1, required for both male and hermaphrodite development in Caenorhabditis elegans. Dev Biol 106, 223-35. (1984).

46. Chan, S. L., Yee, K. S., Tan, K. M. & Yu, V. C. The Caenorhabditis elegans sex determination protein FEM-1 is a CED-3 substrate that associates with CED-4 and mediates apoptosis in mammalian cells. J Biol Chem 275, 17925-8. (2000).

47. Chan, S. L. et al. F1Aalpha, a death receptor-binding protein homologous to the Caenorhabditis elegans sex-determining protein, FEM-1, is a caspase substrate that mediates apoptosis. J Biol Chem 274, 32461-8. (1999).

48. Roth, K. A. Caspases, apoptosis, and Alzheimer disease: causation, correlation, and confusion. J Neuropathol Exp Neurol 60, 829-38. (2001).

49. De Girolami, U. & Feany, M. B. Degenerative diseases of the cerebellum. in Pathology of the Aging Human Nervous System (eds. Duckett, S. & de la Torre, J. C.) 322-359 (Oxford University Press, New York, N. Y., 2001).

50. Imbert, G. et al. Cloning of the gene for spinocerebellar ataxia 2 reveals a locus with high sensitivity to expanded CAG/glutamine repeats. Nat Genet 14, 285-91. (1996).

51. Pulst, S. M. et al. Moderate expansion of a normally biallelic trinucleotide repeat in spinocerebellar ataxia type 2. Nat Genet 14, 269-76. (1996).

52. Sanpei, K. et al. Identification of the spinocerebellar ataxia type 2 gene using a direct identification of repeat expansion and cloning technique, DIRECT. Nat Genet 14, 277-84. (1996).

53. Verkerk, A. J. et al. Identification of a gene (FMR-1) containing a CGG repeat coincident with a breakpoint cluster region exhibiting length variation in fragile X syndrome. Cell 65, 905-14. (1991).

54. Zhang, Y. Q. et al. Drosophila fragile X-related gene regulates the MAP1B homolog Futsch to control synaptic structure and function. Cell 107, 591-603. (2001).

55. Inoue, Y. H. et al. Orbit, a novel microtubule-associated protein essential for mitosis in Drosophila melanogaster. J Cell Biol 149, 153-66. (2000).

56. Lemos, C. L. et al. Mast, a conserved microtubule-associated protein required for bipolar mitotic spindle organization. Embo J 19, 3668-82. (2000).

57. Sokol, N. S. & Cooley, L. Drosophila filamin encoded by the cheerio locus is a component of ovarian ring canals. Curr Biol 9, 1221-30. (1999).

58. Duff, K. & Rao, M. V. Progress in the modeling of neurodegenerative diseases in transgenic mice. Curr Opin Neurol 14, 441-7. (2001).

59. Goedert, M. et al. Phosphorylation of microtubule-associated protein tau by stress-activated protein kinases. FEBS Lett 409, 57-62. (1997).

60. Reynolds, C. H., Betts, J. C., Blackstock, W. P., Nebreda, A. R. & Anderton, B. H. Phosphorylation sites on tau identified by nanoelectrospray mass spectrometry: differences in vitro between the mitogen-activated protein kinases ERK2, c-Jun-terminal kinase and P38, and glycogen synthase kinase-3beta. J Neurochem 74, 1587-95. (2000).

61. Zhu, X. et al. Activation of p38 kinase links tau phosphorylation, oxidative stress, and cell cycle-related events in Alzheimer disease. J Neuropathol Exp Neurol 59, 880-8. (2000).

62. Zhu, X. et al. Activation and redistribution of c-Jun-terminal kinase/stress activated protein kinase in degenerating neurons in Alzheimer's disease. J Neurochem 76, 435-441. (2001).

63. Boutros, M., Paricio, N., Strutt, D. I. & Mlodzik, M. Dishevelled activates JNK and discriminates between JNK pathways in planar polarity and wingless signaling. Cell 94, 109-18. (1998).

64. Schneider, A., Biernat, J., von Bergen, M., Mandelkow, E. & Mandelkow, E. M. Phosphorylation that detaches tau protein from microtubules (Ser262, Ser214) also protects it against aggregation into Alzheimer paired helical filaments. Biochemistry 38, 3549-58. (1999).

65. Zheng-Fischhofer, Q. et al. Sequential phosphorylation of Tau by glycogen synthase kinase-3beta and protein kinase A at Thr212 and Ser214 generates the Alzheimer-specific epitope of antibody AT100 and requires a paired-helical-filament-like conformation. Eur J Biochem 252, 542-52. (1998).

66. Baumann, K., Mandelkow, E. M., Biemat, J., Piwnica-Worms, H. & Mandelkow, E. Abnormal Alzheimer-like phosphorylation of tau-protein by cyclin-dependent kinases cdk2 and cdk5. FEBS Lett 336, 417-24. (1993).

67. Patrick, G. N. et al. Conversion of p35 to p25 deregulates Cdk5 activity and promotes neurodegeneration. Nature 402, 615-22 (1999).

68. Hanger, D. P., Hughes, K., Woodgett, J. R., Brion, J. P. & Anderton, B. H. Glycogen synthase kinase-3 induces Alzheimer's disease-like phosphorylation of tau: generation of paired helical filament epitopes and neuronal localisation of the kinase. Neurosci Lett 147, 58-62. (1992).

69. Ahlijanian, M. K. et al. Hyperphosphorylated tau and neurofilament and cytoskeletal disruptions in mice overexpressing human p25, an activator of cdk5. Proc Natl Acad Sci USA 97, 2910-5. (2000).

70. Feany, M. B. & Bender, W. W. A Drosophila model of Parkinson's disease. Nature 404, 394-8. (2000).

71. Chin, J. Y. et al. Microtubule-affinity regulating kinase (MARK) is tightly associated with neurofibrillary tangles in Alzheimer brain: a fluorescence resonance energy transfer study. J Neuropathol Exp Neurol 59, 966-71. (2000).

72. Vogelsberg-Ragaglia, V., Schuck, T., Trojanowski, J. Q. & Lee, V. M. PP2A mRNA expression is quantitatively decreased in Alzheimer's disease hippocampus. Exp Neurol 168, 402-12. (2001).

73. Lee, M. S. et al. Neurotoxicity induces cleavage of p35 to p25 by calpain. Nature 405, 360-4. (2000).

74. Tomoda, T., Bhatt, R. S., Kuroyanagi, H., Shirasawa, T. & Hatten, M. E. A mouse serine/threonine kinase homologous to C. elegans UNC51 functions in parallel fiber formation of cerebellar granule neurons. Neuron 24, 833-46. (1999).

75. Jackson, G. R. et al. Polyglutamine-expanded human huntingtin transgenes induce degeneration of Drosophila photoreceptor neurons. Neuron 21, 633-42. (1998).

76. Warrick, J. M. et al. Expanded polyglutamine protein forms nuclear inclusions and causes neural degeneration in Drosophila. Cell 93, 939-49. (1998).

77. Steffan, J. S. et al. Histone deacetylase inhibitors arrest polyglutamine-dependent neurodegeneration in Drosophila. Nature 413, 739-43. (2001).

78. Shulman, J. M., Benton, R. & St Johnston, D. The Drosophila homolog of C. elegans PAR-1 organizes the oocyte cytoskeleton and directs oskar mRNA localization to the posterior pole. Cell 101, 377-388 (2000).

79. Neufeld, T. P., de la Cruz, A. F., Johnston, L. A. & Edgar, B. A. Coordination of growth and cell division in the Drosophila wing. Cell 93, 1183-93. (1998).

80. Spradling, A. C. et al. The Berkeley Drosophila Genome Project gene disruption project: Single P-element insertions mutating 25% of vital Drosophilagenes. Genetics 153,135-77. (1999).

81. Starz-Gaiano, M., Cho, N. K., Forbes, A. & Lehmann, R. Spatially restricted activity of a Drosophila lipid phosphatase guides migrating germ cells. Development 128, 983-91. (2001).

82. Rogge, R. et al. The role of yan in mediating the choice between cell division and differentiation. Development 121, 3947-58. (1995).

83. Rebay, I. & Rubin, G. M. Yan functions as a general inhibitor of differentiation and is negatively regulated by activation of the Ras1/MAPK pathway. Cell 81, 857-66. (1995).

84. Jackson, S. M. et al. dally, a Drosophila glypican, controls cellular responses to the TGF-beta-related morphogen, Dpp. Development 124, 4113-20. (1997).

85. Li, W., Ohlmeyer, J. T., Lane, M. E. & Kalderon, D. Function of protein kinase A in hedgehog signal transduction and Drosophila imaginal disc development. Cell 80, 553-562 (1995).

86. Kiger, J. A., Jr., Eklund, J. L., Younger, S. H. & O'Kane, C. J. Transgenic inhibitors identify two roles for protein kinase A in Drosophila development. Genetics 152, 281-90. (1999).

87. Steitz, M. C., Wickenheisser, J. K. & Siegfried, E. Overexpression of zeste white 3 blocks wingless signaling in the Drosophila embryonic midgut. Dev Biol 197, 218-33. (1998).

88. Connell-Crowley, L., Le Gall, M., Vo, D. J. & Giniger, E. The cyclin-dependent kinase Cdk5 controls multiple aspects of axon patterning in vivo. Curr Biol 10, 599-602. (2000).

89. Ma, E. & Haddad, G. A Drosophila CDK5alpha-like molecule and its possible role in response to O(2) deprivation. Biochem Biophys Res Commun 261, 459-63. (1999).

90. Smyth, K. A. & Belote, J. M. The dominant temperature-sensitive lethal DTS7 of Drosophila melanogaster encodes an altered 20S proteasome beta-type subunit. Genetics 151, 211-20. (1999).

91. Wolff, T. Histological techniques for the Drosophila eye. in Drosophila Protocols (eds. Sullivan, W., Ashburner, M. & Hawley, R. S.) 201-243 (CSHL Press, Cold Spring Harbor, N.Y., 2000).

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

What is claimed is:

1. A substantially pure polypeptide consisting essentially of an amino acid sequence selected from the group consisting of: PPAP2A (SEQ ID NO:1); PPAP2B (SEQ ID NO:2); IHPK1 (SEQ ID NO:3); IHPK3 (SEQ ID NO:4); IHPK2 (SEQ ID NO:5); FLJ20530 (SEQ ID NO:6); DJ434014.5 (SEQ ID NO:7); EAF1 (SEQ ID NO:8); Loc127424 (SEQ ID NO:9); Loc113179 (SEQ ID NO:10); and KIAA0826 (SEQ ID NO:1).

2. An antibody made by the process of administering the polypeptide of claim 1 to an animal capable of producing said antibody.

3. A substantially pure polynucleotide consisting essentially of a nucleotide sequence encoding the polypeptide of claim 1.

4. A substantially pure polynucleotide consisting of a nucleotide sequence encoding the polypeptide of claim 1.

5. A vector comprising a distinct coding element consisting of a sequence identical to that of the polynucleotide of either claim 3 or claim 4.

6. The vector of claim 5, wherein said distinct coding element is operably linked to a promoter.

7. A host cell transformed with the vector of claim 6.

8. An assay for the presence of a Tau-related neurodegenerative condition in a subject comprising:
a) removing a biological sample from said subject;
b) determining the amount of a Tau-related polypeptide in said biological sample, wherein said Tau-related polypeptide is selected from the group consisiting of: PPAP2A (SEQ ID NO:1); PPAP2B (SEQ ID NO:2); IHPK1 (SEQ ID NO:3); IHPK3 (SEQ ID NO:4); IHPK2 (SEQ ID NO:5); FLJ20530 (SEQ ID NO:6); DJ434014.5 (SEQ ID NO:7); EAF1 (SEQ ID NO:8); Loc127424 (SEQ ID NO:9); Loci 13179 (SEQ ID NO:10); and KIAA0826 (SEQ ID NO:11);
c) comparing the results obtained in step b) with those obtained in similar biological samples from a control population;
d) concluding that a Tau-related neurodegenerative condition is present in said patient if the comparison of step c) indicates that said subject has a level of Tau-related polypeptide that is either significantly greater or less than that in said control population.

9. The assay of claim 8, wherein said Tau-related neurodegenerative condition is Alzheimer's disease.

10. The assay of claim 8, wherein the amount of Tau-related polypeptide present in said biological samples is determined using a radioimmunoassay or ELISA.

11. An assay for the presence of a Tau-related neurodegenerative condition in a subject comprising:
a) removing a biological sample from said subject;
b) determining the amount of a Tau-related polypeptide in said biological sample, wherein said Tau-related polypeptide is selected from the group consisiting of: ULK1; ULK2; TAO1; TESK1; TESK2; SCA2; FXR1; FXR2; FMR1; FLNA; FLNB; FLNC; CLASP1; CLASP2; GBF1; and GPC5;
c) comparing the results obtained in step b) with those obtained in similar biological samples from a control population;
d) concluding that a Tau-related neurodegenerative condition is present in said patient if the comparison of step c) indicates that said subject has a level of Tau-related polypeptide that is either significantly greater or less than that in said control population.

12. The assay of claim 11, wherein said Tau-related neurodegenerative condition is Alzheimer's disease.

13. The assay of claim 11, wherein the amount of Tau-related polypeptide present in said biological samples is determined using a radioimmunoassay or ELISA.

14. An assay for screening a test compound for its ability to increase the expression of a gene suppressing Tau-related neurotoxicity said assay comprising:
a) contacting cells, with said test compound;
b) measuring the expression of a gene selected from the group consisting of:
PPAP2A (SEQ ID NO:1); PPAP2B (SEQ ID NO:2); FLJ20530 (SEQ ID NO:6);
DJ434014.5 (SEQ ID NO:7); EAF1 (SEQ ID NO:8); Loc127424 (SEQ ID NO:9); and Loc113179 (SEQ ID NO:10);
c) comparing the expression measured in step b) with expression in control cells treated in a similar manner but not contacted with said test compound.

15. An assay for screening a test compound for its ability to inhibit the expression of a gene enhancing Tau-related neurotoxicity said assay comprising:
a) contacting cells expressing said gene, with said test compound, wherein said gene is selected from the group consisting of: IHPK1 (SEQ ID NO:3); IHPK3 (SEQ ID NO:4); IHPK2 (SEQ ID NO:5); and KIAA0826 (SEQ ID NO:11);
b) measuring the expression of said gene; and
c) comparing the expression measured in step b) with expression in control cells treated in a similar manner but not contacted with said test compound.

16. An assay for screening a test compound for its ability to increase the expression of a gene suppressing Tau-related neurotoxicity said assay comprising:
a) contacting cells, with said test compound;
b) measuring the expression of a gene selected from the group consisting of:
ULK1 and ULK2;
c) comparing the expression measured in step b) with expression in control cells treated in a similar manner but not contacted with said test compound.

17. An assay for screening a test compound for its ability to inhibit the expression of a gene enhancing Tau-related neurotoxicity said assay comprising:
a) contacting cells expressing the gene with said test compound;
b) measuring the expression of a gene selected from the group consisting of: TAO1; TESK1; TESK2; SCA2; FXR1; FXR2; FMR1; FLNA; FLNB; FLNC; CLASP1; CLASP2; GBF1; and GPC5;
c) comparing the expression measured in step b) with expression in control cells treated in a similar manner but not contacted with said test compound.

18. A method of treating a Tau-related neurodegenerative condition in a human comprising administering to said human an effective amount of a DNA sequence encoding a protein that suppresses Tau toxicity, and wherein said protein is selected from the group consisting of: PPAP2A (SEQ ID NO:1); PPAP2B (SEQ ID NO:2); FLJ20530 (SEQ ID NO:6); DJ434014.5 (SEQ ID NO:7); EAF1 (SEQ ID NO:8); Loc127424 (SEQ ID NO:9); and Loc113179 (SEQ ID NO:10); ULK1 and ULK2.

19. The method of claim 18, wherein said Tau-related neurodegenerative condition is Alzheimer's disease.
